(19) United States
(12) Patent Application Publication
(10) Pub. No.: US 2025/0114323 A1
(43) Pub. Date: Apr. 10, 2025

(54) METHODS OF REDUCING THE RISK OF CARDIOVASCULAR EVENTS IN PATIENTS WITH LEFT VENTRICULAR HYPERTROPHY

(71) Applicant: LEXICON PHARMACEUTICALS, INC., The Woodlands, TX (US)

(72) Inventor: Michael John Davies, The Woodlands, TX (US)

(73) Assignee: LEXICON PHARMACEUTICALS, INC., The Woodlands, TX (US)

(21) Appl. No.: 18/905,496

(22) Filed: Oct. 3, 2024

Related U.S. Application Data
(60) Provisional application No. 63/542,927, filed on Oct. 6, 2023.

(51) Int. Cl. A61K 31/351; A61P 9/14

(52) U.S. Cl. CPC A61K 31/351; A61P 9/14

(57) ABSTRACT

Methods of reducing the risk of cardiovascular events in patients with left ventricular hypertrophy without hypertension are disclosed. Preferred methods comprise the administration of a therapeutically effective amount of the dual SGLT1/2 inhibitor sotagliflozin.

This application claims priority to U.S. provisional patent application No. 63/542,927 filed Oct. 6, 2023, the entirety of which is incorporated herein by reference.

### FIELD OF THE INVENTION

This invention relates to methods of reducing cardiovascular events in patients with left ventricular hypertrophy.

### BACKGROUND

Sotagliflozin, a dual inhibitor of sodium-glucose cotransporter 1 and 2 (SGLT1 and SGLT2), has proven effective in reducing the risk of cardiovascular death, hospitalization for heart failure and urgent heart failure visit in adults with heart failure or type 2 diabetes mellitus, chronic kidney disease and other cardiovascular risk factors. INPEFA™ Prescribing Information, May 2023. See, also, Bhatt, D. L., et al., “Sotagliflozin in Patients with Diabetes and Worsening Heart Failure” N. Engl. J. Med., 2020 Jan. 14; 384(2):117-128; Bhatt, D. L., et al., “Sotagliflozin in Patients with Diabetes and Chronic Kidney Disease” N. Engl. J. Med., 2021 Jan. 14; 384(2):129-139;

[symbol]

Left ventricular hypertrophy (LVH) is a condition characterized by an increase in left ventricular mass due to an increase in wall thickness and/or left ventricular cavity enlargement. LVH is present in 15% to 20% of the general population. It is more often prevalent in blacks, the elderly, the obese and in patients with hypertension. Cuspidi C., et al., “Italian Society of Hypertension. Prevalence of left-ventricular hypertrophy in hypertension: an updated review of echocardiographic studies” J Hum Hypertens. 2012 June; 26(6):343-9.

Various clinical conditions that can lead to the development of LVH. The most common of these include: essential hypertension; renal artery stenosis; athletic heart with physiological LVH; aortic valvar stenosis; coarctation of the aorta; hypertrophic cardiomyopathy without or with outflow tract obstruction (HOCM); subaortic stenosis (left ventricular outflow tract obstruction by muscle or membrane); aortic regurgitation; mitral regurgitation; dilated cardiomyopathy; ventricular septal defect; and infiltrative cardiac processes (e.g., amyloidosis, Fabry disease, Danon disease). Id.

Hypertension and aortic valve stenosis are the most common causes of LVH. In both cases, the heart is contracting against an elevated afterload. Another cause is increased filling of the left ventricle inducing diastolic overload, which is the underlying mechanism for eccentric LVH in patients with regurgitant valvular lesions such as aortic regurgitation or mitral regurgitation and also seen in dilated cardiomyopathy. Coronary artery disease also plays a role in the pathogenesis of LVH, as the normal myocardium tries to compensate for tissue that has become ischemic or infarcted. Id.

In adults with type 2 diabetes, LVH may occur in the absence of hypertension, particularly in patients with diabetic cardiomyopathy (DbCM), and potentially in those with hypertrophic cardiomyopathy.

### SUMMARY

This invention is directed, in part, to methods of reducing the risk of a cardiovascular event (e.g., heart attack, heart failure, stroke, myocardial infarction, hospitalization for heart failure or urgent visit for heart failure) in patients with ventricular hypertrophy (LVH), including patients with LVH but without hypertension.

One embodiment of the invention encompasses a method of reducing the risk of a cardiovascular event in a patient, which comprises administering to the patient a prophylactically effective amount of an SGLT2 inhibitor, wherein the patient has LVH without hypertension.

Another embodiment encompasses a method of managing heart failure (e.g., slowing the progression of or managing one or more symptoms of heart failure) in a patient at risk of heart failure, which comprises administering to the patient a prophylactically effective amount of an SGLT2 inhibitor, wherein the patient has LVH without hypertension.

Another embodiment encompasses a method of improving the hazard ratio associated with risk of a cardiovascular event in patients taking an SGLT2 inhibitor, which comprises restricting administration of the SGLT2 inhibitor to patients with LVH without hypertension.

Another embodiment encompasses a method of maximizing the efficacy of an SGLT2 inhibitor administered to patients, which comprises restricting administration of the SGLT2 inhibitor to patients with LVH without hypertension.

The invention also encompasses kits (e.g., containers comprising dosage forms and prescribing information) useful in the methods disclosed herein.

### BRIEF DESCRIPTION OF THE DRAWINGS

FIG. 1 shows that the effect of sotagliflozin on the SCORED study's primary endpoint is consistent in the presence or absence of reported LVH or hypertension at baseline.

### DETAILED DESCRIPTION

This invention is directed, in part, to methods of using sotagliflozin, which is the generic name for (2S,3R,4R,5S,6R)-2-(4-chloro-3-(4-ethoxybenzyl) phenyl)-6-(methylthio) tetrahydro-2H-pyran-3,4,5-triol:

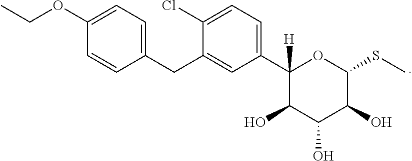 [SMILES: CCOc1ccc(Cc2cc([C@@H]3O[C@H](SC)[C@@H](O)[C@H](O)[C@H]3O)ccc2Cl)cc1]

Sotagliflozin is a dual inhibitor of the sodium glucose cotransporters 1 and 2 (SGLT1 and SGLT2), which is sold in the United States under the tradename INPEFA™ to reduce the risk of cardiovascular death, hospitalization for heart failure and urgent heart failure visit in adults with heart failure or adults with type 2 diabetes mellitus, chronic kidney disease and other cardiovascular risk factors. The compound can be prepared by methods disclosed in the art (e.g., U.S. Pat. No. 7,781,577) and formulated as described, for example, in U.S. patent publication no. US20230218650. Crystalline forms of the compound are disclosed in U.S. Pat. No. 8,217,156.

The terms “sodium glucose cotransporter 2 inhibitor” and “SGLT2 inhibitor” are used interchangeably herein to refer to compounds that inhibit SGLT2, including sotagliflozin, which also inhibits SGLT1. Examples of SGLT2 inhibitors include dapagliflozin, canagliflozin, empagliflozin, sotagliflozin, ipragliflozin, ertugliflozin, tofogliflozin, and luseogliflozin, and pharmaceutically acceptable salts and solvates thereof.

Unless otherwise indicated, the term “combination” when referred to administration of two or more drugs to a patient means that the two or more drugs are both administered (e.g., self-administered) to the patient so that the patient receives the benefit of the two or more drugs (e.g., during a given day). It is not necessary that the two or more drugs be administered at the same time or by the same route of administration.

Unless otherwise indicated, the term “include” has the same meaning as “include but are not limited to,” and the term “includes” has the same meaning as “includes but is not limited to.” Similarly, the term “such as” has the same meaning as the term “such as but not limited to.”

Unless otherwise indicated, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development, and/or duration of the disease or disorder or changing the way that a patient responds to the disease or disorder.

Unless otherwise indicated, the terms “prevent,” “preventing” and “prevention” contemplate an action that occurs before a patient begins to suffer from the specified disease or disorder, which inhibits or reduces the severity of the disease or disorder. In other words, the terms encompass prophylaxis.

Unless otherwise indicated, a “prophylactically effective amount” of a compound is an amount sufficient to prevent a disease or condition or one or more symptoms associated with the disease or condition, prevent its recurrence, or slow or stop its worsening. A “prophylactically effective amount” of a compound means an amount of therapeutic agent, alone or in combination with other agents, which provides a prophylactic benefit in the prevention or retardation of a disease or one or more symptoms thereof.

Unless otherwise indicated, a “therapeutically effective amount” of a compound is an amount sufficient to provide a therapeutic benefit in the treatment or management of a disease or condition, or to delay or minimize one or more symptoms associated with the disease or condition. A “therapeutically effective amount” of a compound means an amount of therapeutic agent, alone or in combination with other therapies, which provides a therapeutic benefit in the treatment or management of the disease or condition. The term “therapeutically effective amount” can encompass an amount that improves overall therapy, reduces, or alleviates the severity of a disease or condition or a symptom thereof, or enhances the therapeutic efficacy of another therapeutic agent.

Unless otherwise indicated, the terms “treat,” “treating” and “treatment” contemplate an action that occurs while a patient is suffering from the specified disease or disorder, which reduces the severity of the disease or disorder, or retards or slows the progression of the disease or disorder.

Unless otherwise indicated, the term “urgent visit” refers to a patient's visit to a heath care provider (e.g., a hospital, clinic, or doctor) resulting from an acute cardiovascular event (e.g., heart attack, stroke) or one or more symptoms associated with a cardiovascular event.

This invention is based on the discovery that patients with left ventricular hypertrophy (LVH) without hypertension who were treated with sotagliflozin to reduce their risk of a cardiovascular event (e.g., heart attack, heart failure, stroke, myocardial infarction, hospitalization for heart failure or urgent visit for heart failure) exhibit a markedly lower hazard ratio than the general population of patients at risk of cardiovascular events. Thus, one embodiment of the invention encompasses a method of reducing the risk of a cardiovascular event in a patient, which comprises administering to the patient a prophylactically effective amount of an SGLT2 inhibitor, wherein the patient has LVH without hypertension.

It is contemplated that in most cases the SGLT2 inhibitor is prescribed (e.g., by a physician) for use in the patient prior to its administration. Particular methods of the invention comprise assessing whether or not a patient has LVH without hypertension before the use of an SGLT2 inhibitor is prescribed.

Another embodiment encompasses a method of managing heart failure (e.g., slowing the progression of or managing one or more symptoms of heart failure) in a patient at risk of heart failure, which comprises administering to the patient a prophylactically effective amount of an SGLT2 inhibitor, wherein the patient has LVH without hypertension.

Another embodiment encompasses a method of improving the hazard ratio associated with risk of a cardiovascular event in patients taking an SGLT2 inhibitor, which comprises administering the SGLT2 inhibitor to patients with LVH without hypertension. In particular methods, administration of the SGLT2 inhibitor is restricted to patients with LVH without hypertension.

Another embodiment encompasses a method of increasing the difference between a hazard ratio associated with risk of a cardiovascular event (e.g., heart attack, heart failure, stroke, myocardial infarction, hospitalization for heart failure or urgent visit for heart failure) in patients taking an SGLT2 inhibitor as compared those on placebo, which comprises restricting administration of the SGLT2 inhibitor to patients with LVH without hypertension.

Another embodiment encompasses a method of improving a hazard ratio associated with risk of a cardiovascular event in patients taking an SGLT2 inhibitor, which comprises recommending administration of the SGLT2 inhibitor to patients with LVH without hypertension.

Another embodiment encompasses a method of improving a hazard ratio associated with risk of a cardiovascular event in patients taking an SGLT2 inhibitor, which comprises restricting administration of the SGLT2 inhibitor to patients with LVH without hypertension.

Another embodiment encompasses a method of maximizing the efficacy of an SGLT2 inhibitor administered to patients at risk of a cardiovascular event, which comprises recommending administration of the SGLT2 inhibitor to patients with LVH without hypertension.

Another embodiment encompasses a method of maximizing the efficacy of an SGLT2 inhibitor administered to patients at risk of a cardiovascular event, which comprises restricting administration of the SGLT2 inhibitor to patients with LVH without hypertension.

Examples of SGLT2 inhibitors include dapagliflozin, canagliflozin, empagliflozin, sotagliflozin, ipragliflozin, ertugliflozin, tofogliflozin or luseogliflozin, or pharmaceutically acceptable salts or solvates thereof. A preferred SGLT2 inhibitor is sotagliflozin.

In particular methods, the SGLT2 inhibitor is administered orally (e.g., as a capsule, caplet or tablet). In specific methods, sotagliflozin is orally administered in an amount of at least 200 mg/day or 400 mg/day.

In particular methods of the invention, the patient has type 2 diabetes. In particular methods of the invention, the patient has experienced heart failure or is at risk of heart failure.

This invention encompasses a kit comprising a container (e.g., a box, bottle, blister tabs) containing individual dosage forms of an SGLT2 inhibitor. In one embodiment, the kit comprises: 1) a container containing at least one solid oral dosage form (e.g., tablet, caplet, or capsule) of an SGLT2 inhibitor; and 2) a label providing prescription information that recommends administration of the SGLT2 inhibitor to patients with LVH without hypertension. In the context of this invention, a label that states that an SGLT2 inhibitor may be administered to patients with LVH without hypertension is considered to “recommend” its administration to those patients.

This invention also encompasses a container containing: 1) at least one solid oral dosage form of an SGLT2 inhibitor, and 2) a label providing prescription information that recommends administration of the SGLT2 inhibitor to patients with LVH without hypertension.

In either the kit or container of the invention, the label may be printed on or affixed to the outside of the container.

Preferred solid dosage forms comprise sotagliflozin (e.g., as a tablet, caplet, or capsule) in an amount of 200 mg or 400 mg.

### EXAMPLES

### Example 1. Study of Patients With Diabetes and Chronic Kidney Disease

A multicenter, double-blind clinical trial (SCORED) was conducted in which patients with type 2 diabetes mellitus (glycated hemoglobin level, ≥7%), chronic kidney disease (estimated glomerular filtration rate, 25 to 60 ml per minute per 1.73 m² of body-surface area), and risks for cardiovascular disease were randomly assigned in a 1:1 ratio to receive sotagliflozin or placebo. The primary end point was changed during the trial to the composite of the total number of deaths from cardiovascular causes, hospitalizations for heart failure, and urgent visits for heart failure.

Of 19,188 patients screened, 10,584 were enrolled, with 5,292 assigned to the sotagliflozin group and 5,292 assigned to the placebo group and followed for a median of 16 months. The rate of primary end-point events was 5.6 events per 100 patient-years in the sotagliflozin group and 7.5 events per 100 patient-years in the placebo group (hazard ratio, 0.74; 95% confidence interval [CI], 0.63 to 0.88; P<0.001). The rate of deaths from cardiovascular causes per 100 patient-years was 2.2 with sotagliflozin and 2.4 with placebo (hazard ratio, 0.90; 95% CI, 0.73 to 1.12; P=0.35). For the original coprimary end point of the first occurrence of death from cardiovascular causes, nonfatal myocardial infarction, or nonfatal stroke, the hazard ratio was 0.84 (95% CI, 0.72 to 0.99); for the original coprimary end point of the first occurrence of death from cardiovascular causes or hospitalization for heart failure, the hazard ratio was 0.77 (95% CI, 0.66 to 0.91). Diarrhea, genital mycotic infections, volume depletion, and diabetic ketoacidosis were more common with sotagliflozin than with placebo.

It was found that in patients with diabetes and chronic kidney disease, with or without albuminuria, sotagliflozin resulted in a lower risk of the composite of deaths from cardiovascular causes, hospitalizations for heart failure, and urgent visits for heart failure than placebo.

### Trial Design

This was a phase 3, randomized, double-blind, placebo-controlled trial that compared sotagliflozin (200 mg once daily, with an increase to 400 mg once daily if unacceptable side effects did not occur) with placebo in patients with type 2 diabetes mellitus, chronic kidney disease, and additional cardiovascular risk; all the patients also received standard-of-care treatments. Randomization was stratified according to criteria for heart failure (ejection fraction of ≤40% documented within the past year or hospitalization for heart failure during the previous 2 years) and geographic region (North America, Latin America, western Europe, eastern Europe, or rest of the world).

### Patients

Persons 18 years of age or older with type 2 diabetes mellitus with a glycated hemoglobin level of 7% or higher, chronic kidney disease (eGFR, 25 to 60 ml per minute per 1.73 m² of body-surface area), and additional cardiovascular risk factors were enrolled. The risk factors consisted of at least one major cardiovascular risk factor in those 18 years of age or older or at least two minor cardiovascular risk factors in those 55 years of age or older. One exclusion criterion was any plan to start an SGLT2 inhibitor during the trial. Written informed consent was obtained from all the patients.

### End Points

The original coprimary end points, assessed in time-to-event analyses, were the first occurrence of a major adverse cardiovascular event (MACE, defined as death from cardiovascular causes, non-fatal myocardial infarction, or nonfatal stroke) and the first occurrence of death from cardiovascular causes or hospitalization for heart failure (HHF). The primary end point was the total number of deaths from cardiovascular causes, hospitalizations for heart failure, and urgent visits for heart failure (UVHF).

The secondary end points were the total number of hospitalizations for heart failure and urgent visits for heart failure; deaths from cardiovascular causes; the total number of deaths from cardiovascular causes, hospitalizations for heart failure, nonfatal myocardial infarctions, and nonfatal strokes; the total number of deaths from cardiovascular causes, hospitalizations for heart failure, urgent visits for heart failure, and events of heart failure during hospitalization; the first occurrence of the composite of a sustained decrease of at least 50% in the eGFR from baseline for at least 30 days, long-term dialysis, renal trans-plantation, or a sustained eGFR of less than 15 ml per minute per 1.73 m² for at least 30 days; deaths from any cause; and the total number of deaths from cardiovascular causes, nonfatal myocardial infarctions, and nonfatal strokes. The use of total events allowed for a single patient to have more than one event contributing to the analysis. Subgroup analyses were prespecified.

### Statistical Analysis

All efficacy analyses followed the intention-to-treat principle. To allow for analyses of total events, competing-risk marginal models for recurrent events that were stratified according to heart-failure criteria and geographic region, with deaths not included in a given end point treated as competing terminal events, were applied to generate hazard ratios with Wald 95% confidence intervals and P values. Proportionality was confirmed by interaction terms between trial-group assignment and the logarithm of time. Event rates were summarized by the number of events per 100 patient-years of follow-up, and accrual of events over time was summarized by cumulative incidence functions. For subgroup analyses, 95% confidence intervals are reported without adjustment for multiple comparisons, and no conclusions can be drawn from these data.

Total events of myocardial infarction and total events of stroke were evaluated post hoc. Testing for differences between the two groups in adverse events was performed post hoc; P values were obtained from Pearson chi-square tests. Change in the eGFR, glycated hemoglobin level, systolic blood pressure, diastolic blood pressure, and weight over time was analyzed post hoc by means of repeated-measures mixed-effects models with absolute change from baseline as the outcome, a random effect for intercept, and fixed effects for trial-group assignment, baseline value, and time. Changes in these outcomes were also jointly modeled with death from any cause to account for competing risk. Data for patients who discontinued the trial were censored for time-to-event end points, and sensitivity analyses were performed, including imputation of the occurrence of events on the date that the patient was last known to be alive and imputation of the same for the sotagliflozin group only.

### Patient Characteristics

A total of 19,188 patients were screened, of whom 10,584 were enrolled; 5,292 were assigned to each trial group. The median age of the patients was 69 years; 44.9% were female, and 82.7% were White. Vital status was available for 99.4% of the patients; 142 (1.3%) did not complete final trial visits, of whom 67 had unknown end-of-trial vital status. The median duration of exposure to sotagliflozin was 14.2 months (interquartile range, 10.3 to 18.9), and the median duration of exposure to placebo was 14.3 months (interquartile range, 10.3 to 18.9). The median duration of follow-up was 16.0 months (interquartile range, 12.0 to 20.3) in the sotagliflozin group and 15.9 months (interquartile range, 11.9 to 20.3) in the placebo group. In the sotagliflozin group, 3944 patients (74.5%) had an increase of the dose from 200 to 400 mg; in the placebo group, 4002 (75.6%) had the dose ostensibly increased. Early discontinuation of the trial regimen for reasons other than death or early trial termination occurred in 578 patients (10.9%) in the sotagliflozin group and 597 patients (11.3%) in the placebo group. Baseline characteristics, shown in Table 1, were similar in the two groups.

TABLE 1
Baseline Characteristics of Patients*

| Characteristic | Sotagliflozin (N = 5292) | | Placebo (N = 2292) | |
|---|---|---|---|---|
| Median age (IQR) - year | 69 | (63-74) | 69 | (63-74) |
| Female sex - no. (%) | 2347 | (44.3) | 2407 | (45.5) |
| Race or ethnic group - no. (%)^{†} | | | | |
| White | 4402 | (83.2) | 4347 | (82.1) |
| Black | 176 | (3.3) | 188 | (3.6) |
| Asian | 317 | (6.0) | 365 | (6.9) |
| American Indian or Alaska Native | 206 | (3.9) | 216 | (4.1) |
| Native Hawaiian or other Pacific Islander | 25 | (0.5) | 15 | (0.3) |
| Multiple | 109 | (2.1) | 95 | (1.8) |
| Unknown | 57 | (1.1) | 66 | (1.2) |
| Median glycated hemoglobin (IQR) - % | 8.3 | (7.6-9.3) | 8.3 | (7.6-9.4) |
| Median body-mass index (IQR) | 31.9 | (28.1-36.2) | 31.7 | (28.0-36.1) |
| Estimated glomerular filtration rate | | | | |
| Median (IQR) - ml/min/1.73 m² | 44.4 | (37.0-51.3) | 44.7 | (37.0-51.5) |
| Distribution - no. (%) | | | | |
| <30 ml/min/1.73 m² | 419 | (7.9) | 394 | (7.4) |
| 30 to <45 ml/min/1.73 m² | 2347 | (44.3) | 2308 | (43.6) |
| >45 ml/min/1.73 m² | 2526 | (47.7) | 2590 | (48.9) |
| Geographic Region - no. (%) | | | | |
| Eastern Europe | 1613 | (30.5) | 1613 | (30.50 |
| Western Europe | 711 | (13.4) | 709 | (13.4) |
| Latin America | 1586 | (30.0) | 1586 | (30.0) |
| North America | 746 | (14.1) | 747 | (14.1) |
| Rest of the World | 636 | (12.0) | 637 | (12.0) |
| Ejection Fraction of ≤40% within past year or | 1054 | (19.9 | 1054 | (19.9) |
| hospitalization for heart failure during previous | | | | |
| 2 years - no. (%) | | | | |
| History of heart failure - no. (%)^{‡} | 1640 | (31.0) | 1643 | (31.0) |
| Ejection fraction of <40% | 505 | (9.5) | 528 | (10.0) |
| Ejection fraction of 40 to <50% | 290 | (5.5) | 291 | (5.5) |
| Ejection fraction of ≥50% | 843 | (15.9) | 824 | (15.6) |
| Ejection fraction unknown | 2 | (<0.1) | 0 | |
| Cardiovascular risk factors - no. (%) | | | | |
| At least one major | 4682 | (88.5) | 4699 | (88.8) |
| No major, at least two minor | 405 | (7.7) | 413 | (7.8) |
| No major and less than two minor | 205 | (3.9) | 180 | (3.4) |
| Previous myocardial infarction - no. (%) | 1051 | (19.9) | 1057 | (22.1) |
| Previous coronary revascularization - no. (%) | 1208 | (22.8) | 1167 | (22.1) |
| Previous stroke - no. (%) | 472 | (8.9) | 474 | (9.0) |
| Urinary albumin-to-creatinine ratio^{§} | | | | |
| Median (IQR) | 74 | (18-486) | 75 | (17-477) |
| Distribution - no. (%) | | | | |
| <30 | 1864 | (35.2) | 1845 | (34.9) |
| 30 to <300 | 1770 | (33.4) | 1819 | (34.4) |
| ≥300 | 1658 | (31.3) | 1628 | (30.8) |
| Median left ventricular ejection fraction (IQR) - %^{‡} | 60 | (51-64) | 60 | (51-65) |
| Median NT-proBNP (IQR) - pg/ml | 196.0 | (75.1-564.6) | 198.1 | (74.6-560.7) |
| Median systolic blood pressure (IQR) - mm Hg | 138 | (127-149) | 139 | (127-149) |
| Median diastolic blood pressure (IQR) - mm Hg | 78 | (70-85) | 78 | (70-85) |
| Any RAAS inhibitor - no. (%)^{¶} | 4705 | (88.9) | 4660 | (88.1) |
| ACE inhibitor | 2009 | (38.0) | 2039 | (38.5) |
| Angiotensin-receptor blocker | 2619 | (49.5) | 2562 | (48.4) |
| Angiotensin receptor-neprilysin inhibitor | 66 | (1.2) | 65 | (1.2) |
| Mineralocorticoid-receptor antagonist | 810 | (15.3) | 776 | (14.7) |
| Beta-blocker - no. (%) | 3310 | (62.5) | 3306 | (62.5) |
| Calcium-channel blocker - no. (%) | 2228 | (42.1) | 2202 | (41.6) |
| Loop diuretic - no. (%) | 1869 | (35.3) | 1867 | (35.3) |
| Other diuretic - no. (%) | 1568 | (29.6) | 1605 | (30.3) |
| Any glucose-lowering medication - no. (%) | 5111 | (96.6) | 5136 | (97.1) |
| Metformin | 2907 | (54.9) | 2955 | (55.8) |
| Sulfonylurea | 1400 | (26.5) | 1486 | (28.1) |
| DPP-4 Inhibitor | 1041 | (19.7) | 1044 | (19.7) |
| Insulin | 3389 | (64.0) | 3333 | (63.0) |
| GLP-1 receptor agonist | 310 | (5.9) | 323 | (6.1) |

*Percentages may not total 100 because of rounding. ACE denotes angiotensin-converting enzyme, DPP-4 dipeptidyl peptidase 4, GLP-1 glucagon-like peptide 1, IQR interquartile range, and NT-proBNP N-terminal pro-brain natriuretic peptide.
^{†}Race and ethnic group were reported by the investigators.
^{‡}Ejection fraction was measured within 1 year before screening or during the screening period.
^{§}The ratio was calculated with albumin measured in milligrams and creatinine measured in grams.
^{¶}Some patients were taking more than one renin-angiotensin-aldosterone system (RAAS) inhibitor.

Of all randomly assigned patients, 19.9% had an ejection fraction of 40% or less within the past year or hospitalization for heart failure during the previous two years, and the median left ventricular ejection fraction was 60% (interquartile range 51 to 65). The median glycated hemoglobin level was 8.3%, median body-mass index (the weight in kilograms divided by the square of the height in meters) was 31.8, median eGFR was 44.5 ml per minute per 1.73 m² (interquartile range, 37.0 to 51.4), and median urinary albumin-to-creatinine ratio was 74 (interquartile range, 17 to 481).

### End Points

There were 701 first and 930 total primary endpoint events. The rates of total primary end-point events were 5.6 and 7.5 events per 100 patient-years in the sotagliflozin and placebo groups, respectively (hazard ratio, 0.74; 95% confidence interval [CI], 0.63 to 0.88; P<0.001).

For the total number of hospitalizations for heart failure and urgent visits for heart failure (first secondary end point), the rate was 3.5 events per 100-patient years in the sotagliflozin group and 5.1events per 100-patient years in the placebo group (hazard ratio, 0.67; 95% CI, 0.55 to 0.82; P<0.001) (Table 2). There was no significant difference between the sotagliflozin and placebo groups in deaths from cardiovascular causes (second secondary end point) (2.2 and 2.4 per 100 patient-years, respectively; hazard ratio, 0.90; 95% CI, 0.73 to 1.12), and the hierarchical analysis stopped at this point. However, results for the composite renal end point did not differ significantly between the two groups, nor did all-cause mortality. The original coprimary end point of the first event of death from cardiovascular causes or hospitalization for heart failure showed a hazard ratio of 0.77 (95% CI, 0.66 to 0.91). The original coprimary end point of the first event of death from cardiovascular causes, nonfatal myocardial infarction, or nonfatal stroke showed a hazard ratio of 0.84 (95% CI, 0.72 to 0.99). For investigator-reported events submitted for adjudication before loss of trial funding, 337 of 501 events (67.3%) in the sotagliflozin group and 460 of 664 events (69.3%) in the placebo group were confirmed on adjudication.

TABLE 2
Primary End Point and Secondary End Points*

| | Events/100 patient-years | | | |
|---|---|---|---|---|
| | (number of events) | | | |
| | Sotagliflozin | Placebo | Hazard Ratio | |
| End Point | (N = 5292) | (N = 5292) | (95% CI)^{†} | P Value |
| Primary end point: total no. of deaths from | 5.6 (400) | 7.5 (530) | 0.74 (0.63-0.88) | <0.001 |
| cardiovascular causes, hospitalizations | | | | |
| for HF, and urgent visits for HF | | | | |
| Major secondary end points, in order of | | | | |
| hierarchical testing | | | | |
| Total no. of hospitalizations for HF and | 3.5 (245) | 5.1 (360) | 0.67 (0.55-0.82) | <0.001 |
| urgent visits for HF | | | | |
| Deaths from cardiovascular causes | 2.2 (155) | 2.4 (170) | 0.90 (0.73-1.12) | 0.35^{‡} |
| Total no. of deaths from cardiovascular | 7.6 (541) | 10.4 (738) | 0.72 (0.63-0.83) | — |
| causes, hospitalizations for HF, | | | | |
| nonfatal myocardial infarctions, and | | | | |
| nonfatal strokes | | | | |
| Total no. of deaths from cardiovascular | 6.4 (453) | 8.3 (589) | 0.76 (0.64-0.89) | — |
| causes, hospitalizations for HF, urgent | | | | |
| visits for HF, and events of HF during | | | | |
| hospitalization | | | | |
| First occurrence of a sustained decrease of | 0.5 (37) | 0.7 (52) | 0.71 (0.46-1.08) | — |
| ≥50% in the eGFR from baseline for | | | | |
| ≥30 days, long-term dialysis, renal | | | | |
| transplantation, or sustained eGFR of | | | | |
| <15 ml/min/1.73 m² for ≥30 days | | | | |
| Deaths from any cause | 3.5 (246) | 3.5 (246) | 0.99 (0.83-1.18) | — |
| Total no. of deaths from cardiovascular | 4.8 (343) | 6.3 (442) | 0.77 (0.65-0.91) | — |
| causes, nonfatal myocardial | | | | |
| infarctions, and nonfatal strokes | | | | |

*The term eGFR denotes estimated glomerular filtration rate; HF denotes heart failure.
^{†}Results are presented as point estimates and 95% confidence intervals unadjusted for multiple comparisons, from which no definite conclusions regarding significant differences can be made.
^{‡}The hierarchical analysis stops after the first P value indicating nonsignificance.

### Adverse Events

There were no significant differences between the two groups in the percentage of patients with adverse events that occurred or worsened during the treatment period or with events leading to withdrawal of the trial regimen. The percentage of patients with a serious adverse event was 23.4% in the sotagliflozin group and 25.2% in the placebo group. Adverse events of special interest that were more common with sotagliflozin than with placebo were diarrhea (8.5% vs. 6.0%; P<0.001), diabetic ketoacidosis (0.6% vs. 0.3%; P=0.02), genital mycotic infections (2.4% vs. 0.9%; P<0.001), and volume depletion (5.3% vs. 4.0%; P=0.003). There were no significant between-group differences in bone fractures, urinary tract infections, severe hypoglycemia, acute kidney injury (2.2% in the sotagliflozin group and 2.1% in the placebo group; P-0.55), or amputations. The percentage of patients in whom hypertension developed was lower with sotagliflozin than with placebo (2.6% vs. 4.1%), whereas the percentage in whom hypotension developed was higher with sotagliflozin than with placebo (2.6% vs. 1.9%; P=0.009).

### Discussion

In this trial, the SGLT1/2 inhibitor sotagliflozin resulted in a lower risk of the composite primary end point of the total number of deaths from cardiovascular causes, hospitalizations for heart failure and urgent visits for heart failure than placebo (5.6 vs. 7.5 events per 100 patient-years). Deaths from cardiovascular causes and renal end points did not differ significantly between the trial groups. Diarrhea, genital mycotic infections, volume depletion, and diabetic ketoacidosis occurred with sotagliflozin.

This trial enrolled patients regardless of urinary albumin-to-creatinine ratio and the trial examined cardiovascular events in a population with a median albumin-to-creatinine ratio of 75 mg/g. This trial also did not require a history of heart failure or reduced ejection fraction at baseline. Despite the low eGFR in the trial population (median, 44.5 ml per minute per 1.73 m²), kidney injury did not differ significantly between the sotagliflozin and placebo groups.

Potential mechanisms of action of SGLT2 inhibition include renal and systemic natriuretic effects, enhanced myocardial energetics, adaptive cellular reprogramming, increased red-cell mass affecting improved oxygen supply, weight loss, reductions in blood pressure and left ventricular hypertrophy, decreases in uric acid, and beneficial effects on endothelial progenitor cells. Sotagliflozin provides some degree of SGLT1 inhibition as well, slowing intestinal glucose absorption and reducing postprandial glycemia. Mendelian randomization data have raised the possibility that SGLT1 inhibition might be associated with decreased rates of cardiovascular events. The SGLT1 blockade may have contributed to increased reports of diarrhea in this trial.

Investigator-defined end-point events were used for all analyses. Approximately 31% of adjudicated heart failure hospitalizations or urgent visits were not confirmed to be primary events, leading to overestimation of the number of events. Furthermore, hospitalization for reasons other than heart failure would remove patients from the risk of a primary end-point event, although fewer total hospitalizations in the sotagliflozin group than in the placebo group (1923 vs. 2094) spanning fewer total patient-years (104 vs. 119) suggest the absence of bias favoring sotagliflozin with respect to the primary end point.

The SGLT1/2 inhibitor sotagliflozin resulted in a lower risk of the composite of deaths from cardiovascular causes, hospitalizations for heart failure, and urgent visits for heart failure than placebo among patients with diabetes mellitus and chronic kidney disease, with or without albuminuria. The results in the sotagliflozin group did not differ significantly from those in the placebo group with respect to rates of death from cardiovascular causes or renal end points.

### Example 2. Analysis of Patients With Left Ventricular Hypertrophy Without Hypertension

A post-hoc analysis of data from the SCORED trial, described in Example 1, was conducted to determine the effects of sotagliflozin on cardiovascular outcomes in patients with reported LVH and no reported hypertension.

In this analysis, patients with reported LVH and no reported hypertension as per the CV Risk Factor Questionnaire (CVRFQ) and medical history [n=418] were identified. LVH patients were identified from an analysis of CVRFQ data, and their hypertension history was determined from medical history data.

The sensitivity analysis was performed by identifying patients as in the primary approach minus those with reported aortic stenosis and those with uncontrolled blood pressure (BP) per CVRFQ [n=361].Aortic stenosis data was obtained from the medical history data. Patients with systolic blood pressure (SBP)>140 mmHg and diastolic blood pressure (DBP)>90 mmHg despite antihypertensive medication (uncontrolled BP) were identified from CVRFQ data. Baseline characteristics of patients with reported LVH and no reported hypertension are shown in Table 3.

TABLE 3
Baseline Characteristics

| | Sotagliflozin | | Placebo | |
|---|---|---|---|---|
| Characteristic | (N = 217) | | (N = 201) | |
| Mean (SD) Age, years | 69 | (9) | 67 | (9) |
| Male, n (%) | 113 | (52) | 107 | (53) |
| Mean (SD) A1C, % | 8.8 | (1.6) | 8.9 | (1.6) |
| Mean (SD) BMI, kg/m² | 32 | (6) | 32 | (6) |
| Mean (SD) SBP, mmHg | 134 | (17) | 134 | (15) |
| Mean (SD) DBP, mmHg | 76 | (10) | 77 | (10) |
| Mean (SD) LVEF, % | 55 | (11) | 56 | (12) |
| LVEF <50% at screening, n (%) | 50 | (23.1) | 41 | (20.4) |
| Mean (SD) eGFR, mL/min/1.73 m² | 47 | (10) | 45 | (9) |
| Median NT proBNP, pg/mL | 292 | | 326 | |
| Any RAAS inhibitor, n (%) | 186 | (76) | 148 | (74) |

Events of special interest in patients with reported LVH and no reported hypertension are presented in Table 4.

TABLE 4
Events of Special Interest

| | Sotagliflozin | Placebo |
|---|---|---|
| Event | (N = 217) | (N = 201) |
| Acute Kidney Injury | 6 (2.8) | 14 (7.0) |
| Bone Fractures | 7 (3.2) | 5 (2.5) |
| Diabetic Ketoacidosis | 1 (0.5) | 1 (0.5) |
| Diarrhea | 19 (8.8) | 10 (5.0) |
| Genital Mycotic Infections | 2 (0.9) | 1 (0.5) |
| Severe Hypoglycemia | 2 (0.9) | 3 (1.5) |
| Any Malignancies of Special Interest | 4 (1.8) | 0 |
| Pancreatitis | 1 (0.5) | 3 (1.5) |
| Urinary Tract Infections | 20 (9.2) | 27 (13.4) |
| Venous Thrombotic Events | 3 (1.4) | 0 |
| Volume Depletion | 10 (4.6) | 7 (3.5) |

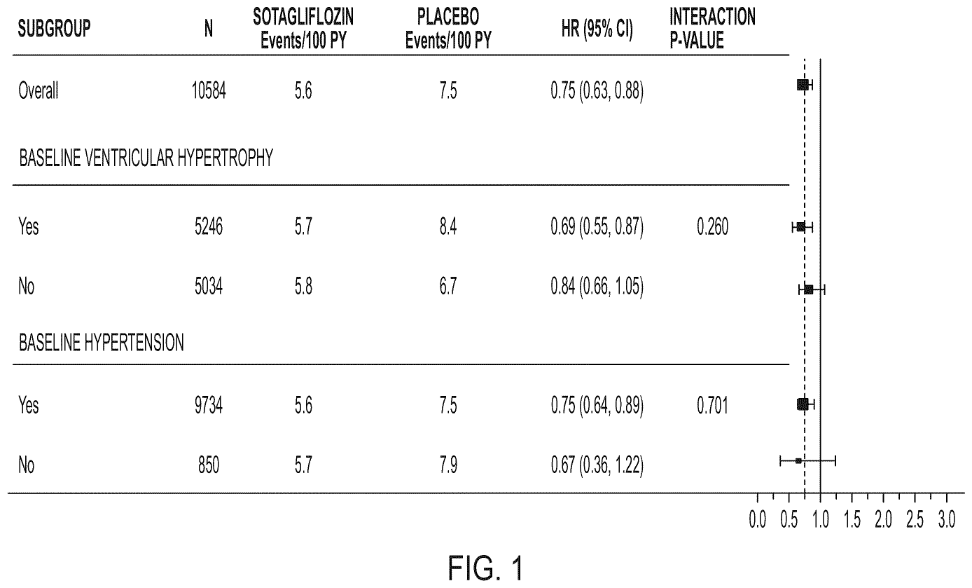

As shown in FIG. 1, the effect of sotagliflozin on the SCORED study's primary endpoint was consistent in the presence or absence of reported LVH or hypertension at baseline.

Event rates were determined for the primary endpoint, three-point major adverse cardiovascular events (MACE: cardiovascular (CV) death, non-fatal myocardial infarction, or non-fatal stroke) and MACE+HHF, and rates for sotagliflozin and placebo were compared. Hazard ratios (HR) and 95% confidence intervals (CI) were calculated for the endpoints. Primary and selected CV endpoints in patients with reported LVH and no reported hypertension are shown in Table 5.

TABLE 5
Primary and Selected Endpoints

| Events per 100 patient- | Sotagliflozin | Placebo | Hazard Ratio |
|---|---|---|---|
| years | (N = 217) | (N = 201) | (95% CI) |
| CV death and HF-related | 5.2 | 9.5 | 0.40 (0.17, 0.92) |
| events (HHF and UVHF) | | | |
| 3-point MACE | 3.1 | 5.3 | 0.52 (0.21, 1.28) |
| 3-point MACE + HHF | 5.5 | 10.2 | 0.46 (0.21, 0.99) |

HHF: hospitalization for heart failure; UVHF: urgent visit for heart failure.

These findings demonstrate that patients with reported LVH and no reported hypertension were at higher risk of the primary endpoint (9.5 events per 100 PY on placebo) compared to the overall cohort (7.5 events per 100 PY on placebo). However, even among these patients, sotagliflozin significantly reduced the risk of CV events.

All publications (e.g., patents and patent applications) cited above are incorporated herein by reference in their entireties.

1-10. (canceled)

11. A method of maximizing the efficacy of an SGLT2 inhibitor administered to patients at risk of a cardiovascular event, which comprises recommending administration of the SGLT2 inhibitor to patients with LVH without hypertension.

12. A method of maximizing the efficacy of an SGLT2 inhibitor administered to patients at risk of a cardiovascular event, which comprises restricting administration of the SGLT2 inhibitor to patients with LVH without hypertension.

13. The method of claim 11, wherein the SGLT2 inhibitor is dapagliflozin, canagliflozin, empagliflozin, sotagliflozin, ipragliflozin, ertugliflozin, tofogliflozin, or luseogliflozin, or a pharmaceutically acceptable salt or solvate thereof.

14. The method of claim 13, wherein the SGLT2 inhibitor is sotagliflozin.

15. The method of claim 11, wherein the patient has type 2 diabetes.

16. The method of claim 11, wherein the patient has experienced heart failure.

17-21. (canceled)

22. The method of claim 14, wherein the sotagliflozin is orally administered in an amount of at least 200 mg/day.

23. The method of claim 22, wherein the sotagliflozin is orally administered in an amount of at least 400 mg/day.

24. The method of claim 12 wherein the SGLT2 inhibitor is dapagliflozin, canagliflozin, empagliflozin, sotagliflozin, ipragliflozin, ertugliflozin, tofogliflozin, or luseogliflozin, or a pharmaceutically acceptable salt or solvate thereof.

25. The method of claim 24, wherein the SGLT2 inhibitor is sotagliflozin.

26. The method of claim 25, wherein the sotagliflozin is orally administered in an amount of at least 200 mg/day.

27. The method of claim 26, wherein the sotagliflozin is orally administered in an amount of at least 400 mg/day.
